(19) United States
(12) Patent Application Publication
(10) Pub. No.: US 2010/0298170 A1
(43) Pub. Date: Nov. 25, 2010

(54) METHODS AND SYSTEMS FOR INTRODUCING FUNCTIONAL POLYNUCLEOTIDES INTO A TARGET POLYNUCLEOTIDE

(76) Inventors: Nicholas Jack Heredia, Mountain House, CA (US); Christine Akiko Hara, Dublin, CA (US); N. Reginald Beer, Pleasanton, CA (US)

Correspondence Address: LLNL/Steinfl & Bruno;John H. Lee, Assistant Laboratory Counsel, Lawrence Livermore National Laboratory, L-703, P.O. Box 808, Livermore, CA, 94551, US

(21) Appl. No.: 12/778,344

(22) Filed: May 12, 2010

Related U.S. Application Data
(60) Provisional application No. 61/177,920, filed on May 13, 2009.

(51) Int. Cl. C40B 50/06; C12P 19/34; C07H 21/00

(52) U.S. Cl. 506/26; 435/91.1; 536/25.3; 435/91.2

(57) ABSTRACT

A method and system is presented for introducing functional polynucleotides into a target polynucleotide using transposons.

### CROSS REFERENCE TO RELATED APPLICATIONS

This application claims priority to U.S. Provisional Application Ser. No. 61/177,920 entitled “In vitro Double Transposition Method for DNA Identification”, filed on May 13, 2009, Docket No. IL12093, the disclosure of which is incorporated herein by reference in its entirety.

### FIELD

The present disclosure relates to methods and systems for introducing polynucleotide fragments into target polynucleotides, in particular, introducing into target polynucleotides using transposons.

### BACKGROUND

The ability to insert polynucleotides into target polynucleotides is desirable. The ability to insert such nucleotides in both a random and/or a controlled method is also desirable. Insertion of known polynucleotides into either known or unknown target polynucleotides offers many possibilities in controlling and/or characterizing the surrounding polynucleotide sequences after insertion. Some potential uses for such insertional events include subsequent sequencing of surrounding polynucleotides, as well as generation of gene knockouts and mutagenesis. Furthermore, the ability to introduce two or more known polynucleotides within a target polynucleotide offers even more possibilities for multiplicity of control and characterization.

### SUMMARY

Provided herein, are methods and systems for introducing polynucleotide fragments into target polynucleotides. In particular, provided herein are methods and systems that in several embodiments allow for introduction of polynucleotides into target polynucleotides using transposons.

According to a first aspect, a method is described for introducing two or more functional polynucleotides into a target polynucleotide. The method comprises inserting at least a first functional polynucleotide of the two or more functional polynucleotides into the target polynucleotide within a first transponson and inserting at least a second functional polynucleotide of the two or more functional polynucleotides into the target polynucleotide within a second transposon, wherein the first functional polynucleotide and the second functional polynucleotide are operably connected.

In another aspect, a method is described for introducing one or more polynucleotides into a target polynucleotide. The method comprises inserting the one or more polynucleotide into the target polynucleotide with one or more transposons, each of the one or more polynucleotides comprised within each of the two or more transposons, the inserting resulting in a targeted polynucleotide containing the one or more polynucleotides. The method further comprises repairing the targeted product in vitro, whereby overhangs are removed and gaps are filled in at the transposon insertion sites.

In another aspect, a system for introducing two or more functional polynucleotides into a target polynucleotide is described. The system comprises at least a first transposon comprising at least a first functional polynucleotide and at least a second transposon comprising at least a second functional polynucleotide; wherein the at least one first functional polynucleotide and the at least one second polynucleotide are operably connected. The system can optionally further comprises a repair solution comprising reagents suitable to repair overhangs and gaps in the transposon insertion sites.

In another aspect, a method is described for in vitro amplification of a target polynucleotide comprised between a first and a second region flanking the target polynucleotide. The method comprises inserting a first functional polynucleotide into the first region within a first transponson, inserting a second functional polynucleotide into the second region within a second transposon thus obtaining a targeted polynucleotide. The method further comprises amplifying the target polynucleotide using at least one pair of primers each pair comprising a first primer able to hybridize the first functional polynucleotide and a second primer able to hybridize the second functional polynucleotide. The method further comprises repairing the targeted polynucleotide in vitro before the amplifying following the insertion of the first and/or the second functional polynucleotides, and possibly also after the amplifying, whereby overhangs are removed and gaps are filled in at the transposon insertion sites

In another aspect, a system is described for in vitro amplification of a target polynucleotide comprised between a first and a second region flanking the target polynucleotide. The system comprises a first transposon comprising a first functional polynucleotide, and a second transposon comprising a second functional polynucleotide. The system further comprises at least one pair of primers each pair comprising a first primer able to hybridize the first functional polynucleotide and a second primer able to hybridize the second functional polynucleotide. The system can further comprise suitable reagents for performing an amplification reaction, in a same or separate composition.

The methods and systems herein described can be used in connection with medical, pharmaceutical, veterinary applications as well as fundamental biological studies and various applications, identifiable by a skilled person upon reading of the present disclosure, wherein investigating immunomodulatory ability and in particular anti-inflammatory ability of a substance is desirable

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure.

FIG. 1 shows a schematic of the double transposition assay for sequencing target DNA in a completely in vitro process, which includes two transposons, two transposases, genomic template, and transposon-associated primers, and produces amplicons for sequencing.

FIG. 2 shows an image of a 1% TBE/agarose gel containing double transposition products. Lane 2 contains a 1 kb ladder (NEB) and lanes 5 and 6 are double transposition reactions. Double brackets are regions where double transposition products were predicted and were excised from gel, purified for repair, and amplified. The upper arrow is linearized LITMUS 28 vector from Sad digest at 2823 bp. The lower arrow is the region corresponding to the two transposons, HyperMu™ <CHL-1>, 1254 bp, and EZ-Tn5198 <T7/Kan-2> transposon, 1248 bp. Lanes 1, 3, 4 and 7 are blank.

FIG. 3 shows an image of a TBE 3% agarose gel of PCR amplicons generated from repaired double transposition DNA excised from the gel in FIG. 2. Lane 1 contains a 1 kb ladder (NEB), Lanes 3-22 are PCR product duplicates of 10 individual replicates (for example, lane 3 and 4 are from a single replicate) using a 1/10 dilution of the extracted material from FIG. 2 as starting template with the four primers T7/Kan-2 FP-1, T7 Kan-2 RP1, MUCHL-1 FP1, MuCHL-1 RP1.

FIG. 4 is a graph of a timecourse study following the single transposition of GPS 1.1 (Tn7) donor into target plasmid DNA Litmus28. Time points were taken every 30 minutes after addition of start solution and halted by incubating at 75° C. for 10 minutes.

FIG. 5 contains graphs of timecourse studies demonstrating transposition of three different donor transposons (GPS 1.1, EZTn5<T7/Kan-2>, and HyperMu <CHL>) into circular target plasmid DNA (Litmus 28) followed by colony counts of aliquots taken from a master mix reaction versus aliquots taken from droplets which were transformed into DH5a E. coli and plated on appropriate antibiotic plates overnight. The aliquots were heat inactivated (10 minutes at 75° C. for GPS1.1 and 10 minutes at 70° C. for HyperMu and EZTn5<T7/Kan-2>) at the indicated times points to halt the transposition reactions. For GPS 1.1 bulk n=2, droplet n=3. For EZTn5<T7/Kan-2>, bulk n=1, droplet n=2. For HyperMu CHL, bulk n=2, droplet n=3. The droplet colony counts for each point in the HyperMu trace was multiplied by 2 for better visualization with the bulk trace.

FIG. 6 shows an image of a monodisperse stream of 65 pl (50 mm diameter) droplets containing the transposon and template reaction mix interspersed in the oil carrier. Images were taken at 2000 Hz by a MotionPro HS-4 CMOS camera with a 4× objective. Channel cross section is elliptical, 150 μm wide by 60 μm deep.

### DETAILED DESCRIPTION

Provided herein are methods and systems using transposons and transposition for the purpose of introducing functional polynucleotide into a target polynucleotide.

As used herein, the terms “transposon” or “transposons” refer to discrete pieces of polynucleotides that can move from one segment of a polynucleotide (donor) to another (target or acceptor) through a process called transposition. Transposons can be made from any type of polynucleotide. Exemplary transposons comprise any form of polynucleotide, such as, but not limited to, mRNA, snRNA, tRNA, rRNA, RNA, cRNA, cDNA or DNA (see e.g. McClintock, 1950; McClintock, 1953)

The term “transposition” or transposition reaction” as used herein indicates a process in which sequences of a polynucleotide are transferred from a donor polynucleotide to an acceptor polynucleotide. Typical transposition reactions make use of transposase enzymes, which recognize the end sequences of transposons and carry out the transfer of the transposon from one polynucleotide molecule to another (Jilk et al., 1996). Typically, in a transposition, a transposase recognizes flanking polynucleotide sequences of a transposon or polynucleotide in a, polynucleotide donor and catalyzes the transfer by a cut and paste mechanism or a replicative transposition mechanism. During an exemplary transposition, a transposase typically cuts the transposon from the donor polynucleotide, selects a region of target polynucleotide, duplicates a region of the target of approximately 5-10 base pair, and severs the bonds between two nucleotides on the leading strand of the target polynucleotide, as well as the bonds between two nucleotides on the lagging strand of the target polynucleotide. The staggered cuts then allow the transposase to insert the transposon into the target polynucleotide. The resulting polynucleotide typically contains the transposon, flanked by short, single-stranded segments of polynucleotides followed by the double-stranded target. The single-stranded regions of the transposon are digested with polymerase. The transposition events can be either specific or random as to where the transposons insert into the target polynucleotide (Craig et al., 1997).

The term “polynucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that is the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Accordingly, the term polynucleotide includes nucleic acids of any length DNA RNA analogs and fragments thereof. A polynucleotide of three or more nucleotides is also called nucleotidic oligomers or oligonucleotides.

The term “functional polynucleotide” as herein indicates a polynucleotide associated to a controllable chemical and/or biological activity. Exemplary controllable activities comprise ability to bind regulatory molecules, such as regulatory proteins and/or regulatory polynucleotide sequences. In particular, functional polynucleotides comprise polynucleotides capable of hybridizing primer sequences under various conditions. In an embodiment, functional polynucleotides comprise polynucleotides able to hybridize with complementary polynucleotide probe sequences that are attached to beads or used in bead-based liquid array formats such as Luminex. In an embodiment, functional polynucleotides comprise polynucleotides able to hybridize with complementary polynucleotide probe sequences that are used for fluorescence detection in real-time applications can be utilized. In various embodiments, functional polynucleotides can be used in conjunction with microarrays, and fluorescent molecules in assays identifiable by a skilled person.

The term “target polynucleotide” as used herein indicates a polynucleotide of interest and can comprise one or more polynucleotides of various origins. Exemplary target polynucleotides comprise bacterial DNA, viral DNA, viral RNA, human DNA and/or human RNA.

In one aspect, a method for introducing polynucleotide fragments into a target polynucleotide is provided that comprises introducing a first functional polynucleotide and a second functional polynucleotide into the target polynucleotide using two transposons, with the first polynucleotide and the second polynucleotide operably connected.

The terms “inserting” or “introducing” as used herein indicate the act of putting or placing an item already in existence and in particular between portions of the item already in existence. In particular, with reference to polynucleotides placement usually requires breaking and formation of covalent linkages between the inserted polynucleotide and the target polynucleotide. Accordingly introducing a first polynucleotide into a target polynucleotide indicates the one or more reactions suitable to place the first polynucleotide within the target polynucleotide through breaking and formation of covalent linkages.

The terms “operably connected” or “operably linked” refer to a functional linkage between two or more elements. In particular, the term “operably linked” or “operably connected” indicates an operating interconnection between biological or chemical activities associated to the two elements. The wording “biological activity” as used herein refers to any activity that can affect the status of a biological molecule or biological entity. A biological molecule can be a protein or a polynucleotide. A biological entity can be a cell, an organ, or a living organism as will be identifiable by a skilled person. A chemical activity indicates to any activity that can affect the chemical status or chemical reactivity of a compound to which the activity refers. Functional linkages between elements in the sense of the present disclosure are identifiable by a skilled person. For example, an operable linkage between a polynucleotide of interest and a regulatory sequence (i.e., a promoter) comprise a functional link that allows for expression of the polynucleotide of interest. Another example of operable linkage is provided by a control sequence ligated to a coding sequence in such a way that expression of the coding sequence is achieved under conditions compatible with the control sequences. Operably linked elements can be contiguous or non-contiguous and can comprise polynucleotides in a same or different reading frame. In an embodiment, each of the operably linked polynucleotide can be comprised within a cassette. The cassette can additionally contain at least one additional gene to be cotransformed into the organism (e.g. a selectable marker gene). One or more additional genes can also be provided on multiple expression cassettes that can further comprise a plurality of restriction sites and/or recombination sites for insertion of other polynucleotides.

In an embodiment, two or more functional polynucleotides can be are operably linked also by way of the specific sites of insertion in the target polynucleotide. For example, in an embodiment, the functional polynucleotides are target of primers for hybridization and the insertion sites allow sequencing of a region of the target polynucleotide comprised between the inserted functional polynucleotides, and/or performance of sequencing and/or further characterization assay of said region using the inserted sequences.

In an embodiment, operably connected functional polynucleotides comprise polynucleotides providing antibiotic resistance genes. In an embodiment, operably connected functional polynucleotides comprise polynucleotide sites inserted between flanking regions of a region of interest in the target polynucleotide designed to be recognized by a transposase. In those embodiments, the inserted sites allow further engineered of the transposons beyond the insertion of the sites and/or other functional polynucleotides (e.g. providing antibiotic resistance)

In an embodiment, the introducing can be performed by inserting two or more separate transposons comprising the functional polynucleotides into the target polynucleotide in a site specific or random fashion using a same or different transposases. Each transposon can be formed by a double-stranded polynucleotide.

In an embodiment, two or more functional polynucleotides can be introduced into a target polynucleotide. In particular, at least a first functional polynucleotide of the two or more functional polynucleotides can be introduced into the target polynucleotide within a first transposon and at least a second functional polynucleotide of the two or more functional polynucleotides can be introduced into the target polynucleotide within a second transposon.

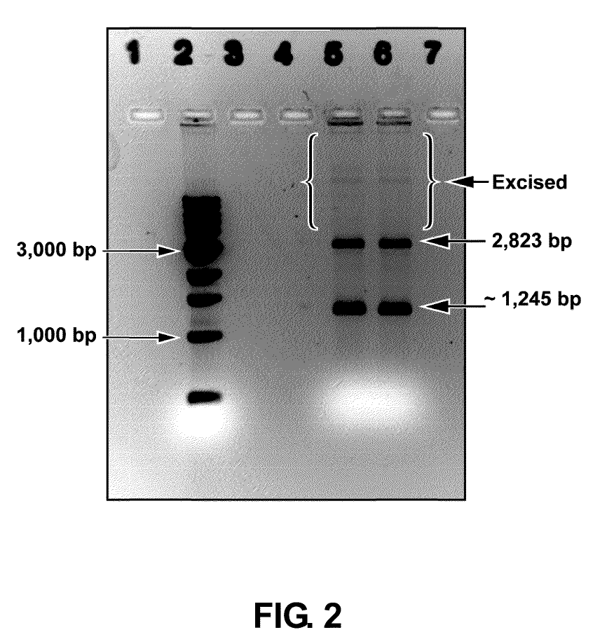

In an embodiment, insertion of a first functional polynucleotide can be specifically targeted or controlled, as shown in Example 3. In another embodiment, the insertion of a first functional polynucleotide can be random or uncontrolled, as shown in Example 2 and FIG. 2.

In an embodiment, the insertion of a second functional polynucleotide can be specifically targeted or controlled, as shown in Example 3. In another embodiment, the insertion of a second functional polynucleotide can be random or uncontrolled, as shown in Example 2 and FIG. 2.

In an embodiment insertion is performed by two or more transposases identifiable by a skilled person each able to insert one transposon into a single target sequence thus providing the targeted product with target immunity towards further transposition from the same transposase. In an embodiment the inserting can be performed by one or more transposases using multiple transposons of the same type to be inserted into a same target sequence with no target immunity for multiple transposition within a same site.

In an embodiment, the inserting can be preceded by a digesting step wherein the target polynucleotide is digested to increase efficiency of the inserting and/or to control location of insertion for at least one of the transposons. The digesting can be performed by contacting the target polynucleotide with an endonuclease or other suitable enzyme for a time and under condition to allow digestion of the target polynucleotide. Specific endonucleases suitable for digesting the target polynucleotides are identifiable by a skilled person. Suitable time and conditions depend on the specific enzyme used and are also identifiable by a skilled person. In an embodiment the digestion can occur at 37° C. Typically, a digestion reaction involves template, an endonuclease and a reaction buffer with incubation. The enzyme can then be heat inactivated at the end of the reaction.

In an embodiment, the target polynucleotide can be formed by sheared polynucleotides, (e.g. with a 200 bp DNA formed starting from a 454 DNA)

In an embodiment, the target polynucleotide can comprise multiple polynucleotides. In some of those embodiments, transposition is performed in a bulk reaction or using droplet instrumentation. In some of those embodiments, the functional polynucleotides are polynucleotides able to hybridize complementary probes (e.g. real time probes) and the method comprises an assay using RT-PCR in a downstream analysis In some of those embodiments, the probes can be designed to perform identification across a family or genus of organisms

In an embodiment, a portion of the transposon sequence can be used as a priming site to sequence a region of the target polynucleotide of interest in a shotgun approach. In an embodiment, at least a portion of a transposon exhibit target immunity, which makes the target polynucleotide immune from further transposition by the same transposon once it has entered the target polynucleotide up to 190 kb from the insertion site (DeBoy and Craig, 1996; Stellwagen and Craig, 1997). In some of those embodiments, the target polynucleotide is linear and the method can comprise a shearing, digesting or otherwise breaking the target polynucleotide before or after the introducing. In some embodiments, large (e.g. from approx >5,000 bp to approx >2,000 Kb or greater although a different number can be identified in view of the experimental design), polynucleotides within the target sequence can be broken or not before the inserting.

In an embodiment, the one or more transposases can be tethered together. The one or more transposases can be tethered together by one or more tethering element such as antibodies, oligonucleotides, peptides, synthetic linkers, biopolymers, a biotinylation/streptavidin linker, and combinations thereof.

In an embodiment, multiple transposases can be tethered together for example, for the purpose of having the transposases insert transposons near each other in the target polynucleotide to be further characterized. In some of these embodiments tethering can be performed with antibodies. The tethering can also include peptides or nucleic acid linkers between multiple same or different antibodies for the purpose of having the transposes insert the transposons near each other in the target polynucleotide. Tethering units can be directly or indirectly linked to the transposases for example via an oligonucleotide, peptide, or synthetic linker or biopolymer. In another embodiment, the transposases can be biotinylated and tethered together via a streptavidin linker. Embodiments where the transposases are tethered allow greater control of the spacing between the transposons inserted into the target polynucleotide and; thus, improve the ability to amplify or otherwise characterize the region of the target polynucleotide effectively.

In another embodiment, the tethering of transposases using antibodies allows the property of target immunity of some transposons to be used to an advantage so that if the two tethered transposases have the property of target immunity, the target polynucleotide would only have one copy of each of the two different transposons inserted into it. This property would decrease the chance of error in subsequent sequencings steps of the amplified polynucleotide, as there would be only one region amplified on the target polynucleotide.

In various embodiments, methods herein described allow repairing of the polynucleotides following transposition. Typically, during the transposition reaction, the target polynucleotide is nicked/cut by the transposase. A short segment of target polynucleotide is then repeated. The target is then nicked to the left of the sequence on the upper strand while the polynucleotide is nicked to the right of the sequence on the lower strand. This series of events results in staggered or sticky ends. The transposon is then incorporated into the polynucleotide.

In an embodiment, one or more functional polynucleotides can be introduced into the target polynucleotide with one or more transposons, each of the one or more functional polynucleotides comprised within each of the two or more transposons, the introduction of the polynucleotides resulting in a targeted polynucleotide containing the one or more functional polynucleotides. The method further comprises repairing the targeted product in vitro, whereby overhangs are removed and gaps are filled in at the transposon insertion sites.

In particular, the repairing can be performed by contacting the targeted product with one or more enzymes, and suitable reagents to digest overhangs of the transposon and polymerizing a complementary strand for the single stranded target polynucleotide. In an embodiment, the enzyme and suitable reagents comprise a polymerase with exonuclease activity, such as E. coli polymerase 1. In an embodiment, the enzyme and suitable reagents comprise a polymerase and kinase such as T4 DNA polymerase to remove overhangs and T4 polynucleotide kinase used to phosphorylate the 5′ ends for subsequent blunt-end ligation reactions.

The term “overhang” as used herein indicates a stretch of unpaired nucleotides in the end of a polynucleotide molecule. These unpaired nucleotides can be in either strand, creating either 3′ or 5′ overhangs. Such removal/filling in of overhangs and/or gaps can be termed “lesion repair” and one aspect is described in Example 4. The lesion repair can be carried out by any method that would be understood by those skilled in the art, such as by using enzymes, polymerases or kits.

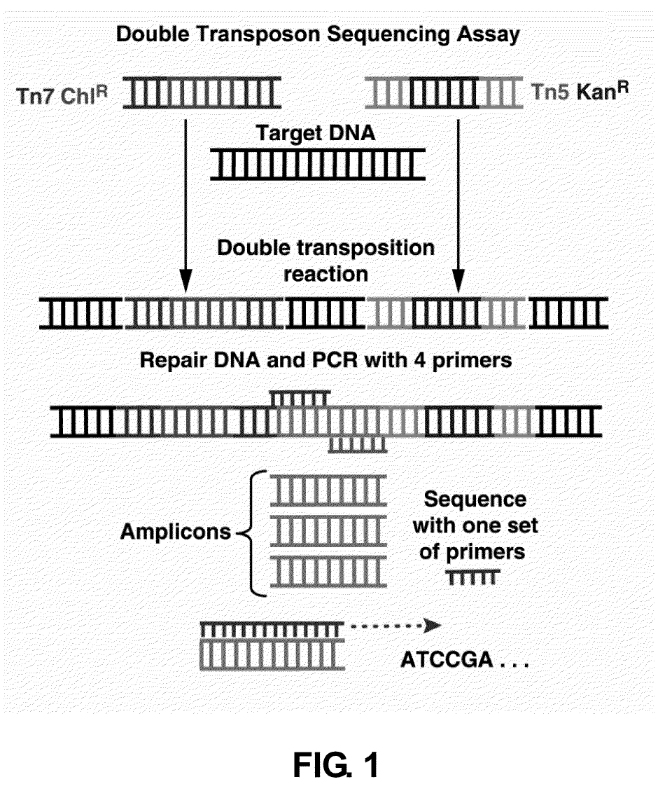

Exemplary steps of methods wherein inserted transposons are repaired are described in Example 1 and depicted in FIG. 1.

In some embodiments, in vitro double transposition can be considerably faster and/or simpler than the conventional use of transposition kits, as described in Example 7 and Table 2. In particular, embodiments where in vitro double transposition is performed do not require transformation of bacteria for repairing transposition lesions of the targeted product.

In addition to simplifying experimental procedures, time can be saved by conducting the reactions by in vitro methods presented herein. Table 2 shows a time comparison in hours between the double transposition assay and the conventional use of transposons for sequencing unknown DNA. The various steps are listed for each system, as well as the approximate time required for performing each step. In vitro double transposition is considerably faster than the conventional use of transposition kits.

In an embodiment of methods herein described, the targeted product obtained following insertion of one or more functional polynucleotides and, optionally repairing, can be further characterized. Characterization of the targeted product can be performed by techniques and procedures such as by gel purification, PCR, high-throughput means, and combinations thereof, and additional techniques identifiable by a skilled person.

An exemplary characterization can be performed by isolating the target and subsequently amplifying the target (e.g. by PCR) as shown in Example 5. Exemplary procedures to perform the isolating comprise Luminex, bead-based liquid array identification, real-time PCR, and microarrays. Additional procedures to perform the isolating comprise sorting individual droplets of a picoliter size of the reaction mixture via instrumentation, and separating the targeted product based on charge and/or mass.

In particular, in an embodiment, the sorting can be performed based on fluorescence or other labeling. In the embodiment fluorescently labeled nucleotides are comprised in reaction mixtures for the inserting and/or the repairing. Successful transposition can be detected through detection of the labels following transposition.

The terms “label” and “labeled molecule” as used herein as a component of a complex or molecule refer to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence the wording and “labeling signal” as used herein indicates the signal emitted from the label that allows detection of the label, including but not limited to radioactivity, fluorescence, chemolumiescence, production of a compound in outcome of an enzymatic reaction and the likes.

The term “detect” or “detection” as used herein indicates the determination of the existence, presence or fact of a target or signal in a limited portion of space, including but not limited to a sample, a reaction mixture, a molecular complex and a substrate. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

In an embodiment, wherein transposons comprise an internal label such as biotins, the isolating can be performed by contacting the targeted product with streptavidin; detecting the biotin-streptavidin complexes and separating said complexes. In another embodiment, a label such as fluorophore can be bound to the internal biotin, such as in SAPE. In another embodiment, the transposition products can be isolated by hybridization arrays, where the hybridized polynucleotide is the inserted transposon or a portion thereof.

In another embodiment, the sorting can be performed using electric field on a microfluidic platform. Droplets that contain a transposed item can be directed to a separate holding coil while droplets containing no sample are directed to waste. One then has the ability to sort, based on a label such as fluorescence, either after the transposition reaction occurs or after the amplification of the transposed polynucleotide occurs.

In an embodiment, procedures suitable to perform the isolating can be performed through high-throughput, automated, small reaction volume, robotic controlled reactions. These reactions can take place in microtiter and/or microwell plates, which isolate very small reaction volumes, and allow manipulation of the double transposition products by automated machinery

A further exemplary characterization procedure comprises sequencing, as depicted in Example 6. The sequencing can comprise pyrosequencing and/or mass spectrometry.

In an embodiment methods herein described can be used for inserting of so-called ‘universal’ sequences (sequences with an arbitrary length and arbitrary number of unique units) to act as priming sites prior to high-throughput sequencing ((Margulies et al., 2005).).

In an embodiment, methods described herein can be used to identify both known and unknown organisms in the absence of specific sequence information, in particular in connection with the characterization of complex biological samples.

In particular, in an embodiment, methods using polynucleotide transposition to introduce and utilize universal sequences in a wholly in vitro technique for polynucleotide identification

Transposition can offer advantages over current sequencing technologies in keeping the genome of unknown targets intact and reducing the enzymatic steps needed to prepare the DNA library.

In an embodiment a method is described for in vitro amplification of a target polynucleotide comprised between a first and a second region flanking the target polynucleotide. The method comprises inserting a first functional polynucleotide into the first region within a first transponson, inserting a second functional polynucleotide into the second region within a second transponson. The method further comprises amplifying the target polynucleotide using at least one pair of primers each pair comprising a first primer able to hybridize the first functional polynucleotide and a second primer able to hybridize the second functional polynucleotide. The method can further comprise repairing the targeted product in vitro after the inserting before and/or after the amplifying, whereby overhangs are removed and gaps are filled in at the transposon insertion sites.

In embodiments wherein primers can be used to hybridize the inserted functional polynucleotides in subsequent PCR reactions, each transposon can have a primer that can hybridize to its leading strand as well as a primer that binds to the lagging strand. Thus, when both transposons are inserted there can be four primer binding sites and amplification can be performed as long as the transposons land within a few kilobases of each other. In an embodiment, the transposition reaction can be random and performed with multiple targets per reaction, and multiple products can be produced. For example, targeted product can have two transposons 500 bases apart, as well as 50 bases apart, 200 bases apart or 1,000 bases apart.

In an embodiment, a system for in vitro amplification of a target polynucleotide comprised between a first and a second region flanking the target polynucleotide. The system comprises a first transposon comprising a first functional polynucleotide, a second transposon comprising a second functional polynucleotide, and at least one pair of primers, each pair comprising a first primer able to hybridize the first functional polynucleotide and a second primer able to hybridize the second functional polynucleotide.

In an embodiment, the methods and systems can be performed and incorporated into a high throughput, automated microfluidic system with the entire process performed in vitro, using procedures such as the ones exemplified in Example 8.

The term “microfluidic” as used herein refers to a component or system that has microfluidic features e.g. channels and/or chambers that are generally fabricated on the micron or sub-micron scale. For example, the typical channels or chambers have at least one cross-sectional dimension in the range of about 0.1 microns to about 1500 microns, more typically in the range of about 0.2 microns to about 1000 microns, still more typically in the range of about 0.4 microns to about 500 microns. Individual microfluidic features typically hold very small quantities of fluid, e.g. from about 100 femtoliters to about 1 milliliter, more typically from about 1 picoliter to about 500 microliters, still more typically from about 10 picoliters to about 50 microliters, or yet more typically from about 50 picoliters to about 25 microliters.

In an embodiment of the methods and system herein described, in vitro amplification, repair, and/or hybridization can be performed at any scale on the benchtop in standard formats.

In an embodiment of the methods and system herein described, in vitro amplification, repair, and/or hybridization can be performed at any scale within a bulk emulsion with compartmentalized reactors.

In an embodiment of the methods and system herein described, in vitro amplification, repair, and/or hybridization can be performed at any scale with a microfluidic arrangement wherein reagents are comprised on magnetic or non-magnetic beads

In an embodiment of the methods and system herein described, in vitro amplification, repair, and/or hybridization can be performed at any scale with a microfluidic arrangement wherein transposons, primers and other reagents are comprised in monodisperse or polydisperse emulsions

In an embodiment of the methods and system herein described, in vitro amplification, repair, and/or hybridization can be performed at any scale with a microfluidic and/or robotic arrangement utilizing microwells, microarrays, or micro-titer plates enabling sample discretization and transposon operation on volumes of any desirable scale.

In an embodiment of the methods and system herein described, in vitro amplification, repair, and/or hybridization can be performed at any scale with a microfluidic arrangement wherein successful droplets or compartments can be detected (e.g. optically detected) utilizing functional probes and subsequently selected or sorted for additional repair, hybridization, or amplification steps depending on the desired assay.

In an embodiment of the methods and system herein described, in vitro amplification, repair, and/or hybridization can be performed at any scale with a microfluidic arrangement wherein successful droplets or compartments can be detected, and in particular optically observed, utilizing functional probes and subsequently selected or sorted for additional repair, hybridization, or amplification steps depending on the desired assay, wherein the resulting product can be used for sequencing, and in particular for oligonucleotide sequencing.

In an embodiment of the methods and system herein described, in vitro amplification, repair, and/or hybridization is performed at any scale in any format allowing high sample throughput, continuous product sorting, and reaction monitoring

In an embodiment, a microfluidic system can be configured to heat-inactivate on the system. In an embodiment, the microfluidic system comprises a monodispersion microfluidic platform. In an embodiment, the microfluidic system comprises a bulk emulsion platform, as well as robotic, high throughput nanoscale reactions. In an embodiment, the microfluidic system comprises paper-based microfluidic platforms, as described in (Martinez et al 2008).

As disclosed herein, the transposons and/or functional polynucleotides herein described can be provided as a part of systems to perform any assay, including any of the methods described herein. In some embodiment, a system according to the present disclosure can comprises at least a first transposon comprising at least a first functional polynucleotide and at least a second transposon comprising at least a second functional polynucleotide; wherein the at least one first functional polynucleotide and the at least one second polynucleotide are operably connected. The system can optionally further comprises a repair solution comprising reagents suitable to repair solution repairs overhangs and gaps in the transposon insertion sites.

In an embodiment, a system herein described can further comprise at least one pair of primers each pair comprising a first primer able to hybridize the first functional polynucleotide and a second primer able to hybridize the second functional polynucleotide

The systems herein described can be provided in the form of kits of parts. In a kit of parts, the first polynucleotide, second polynucleotide, reagents suitable for perform the repairing and other reagents to perform the method can be comprised in the kit independently. The first polynucleotide, second polynucleotide, repair composition including reagents suitable to perform the repairing reactions and other reagents can be included in one or more compositions, and each component and reagent can be in a composition together with a suitable vehicle.

Additional components can include dNTPs, divalent ions, buffers such as Tris-HCl, an energy source like NAD+, plasmids as control target polynucleotides, labeled polynucleotides, labeled antibodies, labels, microfluidic chip, reference standards, and additional components identifiable by a skilled person upon reading of the present disclosure.

In particular, in an embodiment of kit used for in vitro amplification included nucleotides, MgSO4, PCR buffer and a Taq polymerase. Different types of polymerases can be used as will be understood by a skilled person. Single components are commercially available.

The components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

Further details concerning the identification of suitable polynucleotides and polynucleotide constructs or auxiliary agents of the compositions, and generally manufacturing and packaging of the kit, can be identified by the person skilled in the art upon reading of the present disclosure.

### EXAMPLES

The methods and systems herein disclosed are further illustrated in the following examples, which are provided by way of illustration and are not intended to be limiting.

The following materials and methods were used for all the methods and systems for in vitro transposition for DNA identification exemplified herein.

### Example 1

### In vitro Double Transposition for Use in a Sequencing Reaction

As shown in FIG. 1, the steps for in vitro double transposition to generate amplicons of target DNA for use in a sequencing reaction involve (1) transposition into target DNA, (2) followed by in vitro DNA repair to remove overhangs and fill in gaps at the transposon insertion site, (3) amplification of the region between the two transposons through a PCR reaction with four primers from the two transposon systems, (4) treatment of the PCR reaction with ExoSAP-IT™ to remove PCR primers and inactivate dNTPs, and (5) sequencing of the amplicons generated from the PCR reaction using a forward and reverse primer set from only one of the transposon systems used.

### Example 2

### Generation of Double Transposition DNA Products through Random Insertion

Three commercially available kits were used to transpose either one or two different transposons into target DNA: EZ-Tn5™ <T7/KAN-2>, HyperMu™ CHL (both from Epicentre Biotechnologies), and GPS1.1 transprimer donor (New England BioLabs). The target DNA was either circular or linearized Litmus 28 plasmid. EZ-Tn5™ <T7/KAN-2> and HyperMu™ are derivatives of the Tn5 and Mu transposons and harbor the antibiotic resistance markers for kanamycin and chloramphenicol, respectively. The GPS-1 Genome Priming System™ is a Tn7 based system from which GPS1.1 transprimer donor was used, which bears a kanamycin resistant marker within the transposon. Both EZ-Tn5™ <T7/KAN-2> and HyperMu™ inserted into target DNA under similar reaction conditions and were used for double transposition. Double transposition reactions have also been performed using the GPS1 and EZ-Tn5™ <T7/KAN-2> transposons, which required the addition of a start solution to the reaction (data not shown).

The 20 μl double transposition reaction mixture was made with 2 μl of 10× HyperMu™ reaction buffer, 10 μl of template using the 2823 bp Litmus 28 (New England Biolabs) linearized by digestion with Sad (New England Biolabs) at a concentration of 29.5 ng/μl, 2 μl of HyperMu™ transposon, 1 μl of EZ-Tn5™ <T7/KAN-2> transposon, 1 μl of HyperMu™ Mu A transposase, 1 μl EZ-Tn5™ transposase, and 3 μl of nuclease-free water (Ambion). The reaction was incubated in a thermocycler at 37° C. for one hour. The reaction products were run on a precast 1% TBE/agarose gel pre-stained with EtBr (BioRad) and double transposition products were excised from the gel for further manipulation or analysis.

Exemplary results of double transposition reactions with linearized Litmus 28 plasmid DNA as the target using the two transposons, Mu and Tn5, and their corresponding two transposes are illustrated in FIG. 2.

In particular, FIG. 2 shows an image of a 1% agarose gel where regions where double transposition products are predicted, are indicated in brackets, The bracketed region was gel purified, repaired, and used as template in subsequent PCR reactions with four primers from the two transposon systems as illustrated in detail in the following example 4. The large excised region was chosen to capture the various sizes of transposition products produced by the random insertion of the transposon. The dimensions of the regions to be excised were determined by knowing the initial size of the linearized plasmid and the known sizes of the transposons. The region on the gel to be excised should have a mass greater than the known plasmid with the size of both transposons added. “Known size” is referring to the number of bases or nucleotides per plasmid or transposon. The size of the target and transposons was provided in the literature that came with each kit. In order to visualize the transposons, plasmid DNA and successful transposon reactions, gel electrophoresis was used, as seen in FIG. 2. In lane 2, there is a DNA step ladder that indicates how far a DNA sample of “X’ base pairs should have migrated down the gel. Using the mass ladder, it is shown that the transposons migrate to ˜1245 bp and the transposon migrates to ˜2823 bp. Anything larger than 2823 bp should contain at least one transposon. In the illustration of FIG. 2 the inserted transposons migrate slower in the gel because they are nicked.

It is also possible to fraction the regions based upon size exclusion spin columns. For any given reaction, the expected size of the target template and transposons can be calculated. If spin columns are available that can discriminate between the fragments of individual transposons and transposed target then one is able to fraction out potential target candidates. For example, if the transposons are 200 bp each and targets that are at least 1000 bp are of interest, one can select a spin column that fractions fragments under 500 bp. Thus the target (both transposed and not) would be separated from the individual transposons. Further dilutions and PCR reactions can be performed to determine which targets were successfully transposed.

### Example 3

### Generation of Double Transposition DNA Products through Site Specific Insertion—Prophetic

Transposition using methods and systems herein described can also be performed using site-specific transposition. In particular the introducing of one or more functional polynucleotides can be performed using site-specific transposition as shown in (Marcus, J. M., (2003) herein incorporated by reference in its entirety.

### Example 4

### Double Transposition Lesion Repair

Regions from agarose gel generated where transposons insertion are expected generated in outcome of the experiments of Example 2, were gel purified, repaired and amplified according to procedures exemplified in the present example.

The gel purified double transposition reaction products produced following experiments detailed in Examples 2 and 3 contain DNA lesions consisting of overhangs from the transposition process and these were repaired using PreCR™ repair mix (New England BioLabs) with the modification of adding E. coli DNA polymerase I (Epicentre Biotechnologies DP021K). The PreCR™ repair kit is designed to repair nicks and/or damage in DNA, such as apurinic/apyrimidinic sites, as well as fill in any gaps; however, it does not have exonuclease activity to remove the single stranded overhangs that are formed in the transposition process after the transposon has inserted into the target DNA.

The lesions are mismatched and must be removed, which is conventionally done by transforming the transposition products into bacteria. To maintain compatibility with an autonomous microfluidic platform, the goal is to avoid transformation into bacteria and perform the entire reaction in vitro.

To achieve this E. coli polymerase I was added, which has exonuclease activity to the PreCR™ repair mix to remove these overhangs, allowing PreCR™ to fill in the gaps and seal the nicks, repairing the DNA damage in vitro. It will be understood by those skilled in the art that other polymerases can be used as long as they have exonuclease activity. The PreCR™ repair reaction was made of 4.60 ThermoPol reaction buffer, 18.4 μl dNTPs at 250 μM , 0.5 μl of 100×NAD+, 1 μl E. coli Polymerase I, 1 μl PreCR™ repair mix, and 20.5 μl (4.2 ng/μl) gel purified double transposed Litmus28 DNA and was incubated at 37° C. for 20 minutes. Success of repair can be determination by quantitatively detecting an amplification product.

### Example 5

### Double Transposition Amplification

The purified and repaired regions from agarose gel of Example 2, where used as template in subsequent PCR reactions with four primers from the two transposon systems.

In particular, 1 μl of the post-PreCR™ reaction was used as template for a PCR reaction as follows: 12.5 μl SuperScript III (Invitrogen), 0.5 μl of each of the four transposon primers T7/KAN-2 FP-1, T7/KAN-2RP-1, MUCHL-1 FP-1, and MUCHL-1 RP-1, each at a concentration of 10 μM, 1 μl Platinum Taq (Invitrogen), 0.8 μl of 50 mM MgSO₄ (final concentration of 1.6 mM), 7.70 of nuclease-free water, and 1 μl of post-PreCR™ template. The PCR reaction was thermocycled according to the Superscript III manufacturer's protocol. The PCR reaction was then treated with exonuclease I and shrimp alkaline phosphatase (ExoSAP-IT™, USB Corporation) at a ratio of 2 μl of ExoSAP-IT™ for every 50 of post-PreCR reaction. ExoSAP-IT™ was added to remove the PCR primers and nucleotides in preparation for DNA sequencing.

The samples were incubated with ExoSAP-IT™ at 37° C. for 15 minutes, and followed by a heat inactivation step at 80° C. for 15 minutes. The samples were finally loaded in a TBE gel agarose for visualization.

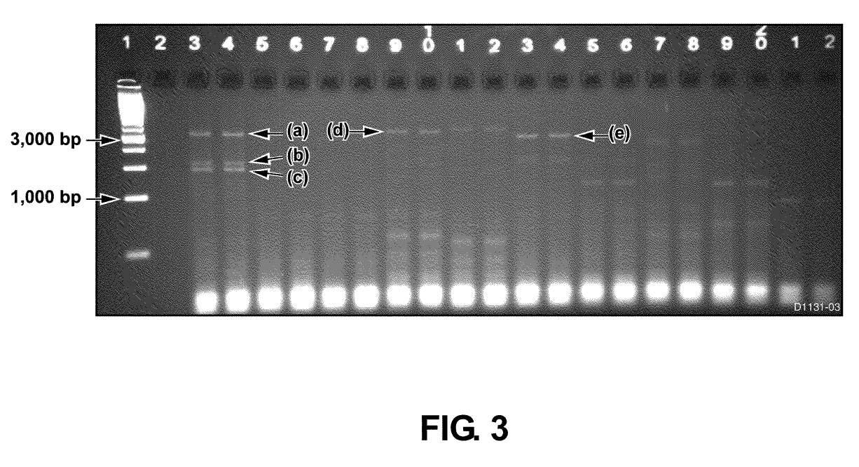

The results illustrated in FIG. 3 indicate that double transposition took place. FIG. 3 is a representative gel image showing the amplicons produced from PCR with the four primers. Different results are obtained in nonreplicate lanes because of the random insertion locations of the transposons. Five of the bands (a-e) were excised from the gel in FIG. 3 and purified for sequencing experiments using the sequencing primers MUCHL-1 FP-1 and MUCHL-1 RP-1. The results and related characterization are illustrated in Table 1 (see in particular Experiment 1) together with results of transposons insertions and amplifications performed with other samples.

### Example 6

### Double Transposition Sequencing and Characterization

Amplicons were extracted from 3% TBE agarose gels and purified using the Qiaquick Gel Extraction Kit (Qiagen). The purified DNA was quantified using a NanoDrop ND-1000 spectrophotometer and 10 ng of purified DNA was used in the BigDye® Terminator v3.1 Sequencing Kit reaction (Applied Biosciences) with 3.2 pmol of primers (identified in Table 1). The reactions were run for 50 cycles and analyzed using an ABI 3130x1 genetic analyzer. The experimental and actual sequences were aligned and compared with BLAST (bl2seq) (Tatusova and Madden (1999)).

In particular, Table 1 reports the primer sets used in sequencing, the percent identity match with Litmus 28, the number of gaps, and the E value, which represents the probability due to chance that another alignment can have greater similarity to the known sequence.

TABLE 1

| | | |
|---|---|---|
| Sequencing | Litmus 28 | |
| Primer Set | Identities | Gaps |

| | | | | |
|---|---|---|---|---|
| Experiment 1 | MUCHL-1 | 71/84 | (84%) | 0 |
| | FP-1/PRP1 | | | |
| Experiment 2a | MUCHL-1 | 202/206 | (98%) | 1/206 |
| | FP-1/RP1 | | | |
| Experiment 2b | MUCHL-1 | 160/174 | (91%) | 5/174 |
| | FP-1/RP1 | | | |
| Experiment 2c | MUCHL-1 | 47/52 | (90%) | 0/52 |
| | FP-1/RP1 | | | |
| Experiment 2a | T7/KAN-2 | 107/134 | (79%) | 15/134 |
| | FP-1/RP-1 | | | |
| Experiment 2b | T7/KAN-2 | 193/207 | (93%) | 7/207 |
| | FP-1/RP-1 | | | |
| Experiment 2c | T7/KAN-2 | 38/51 | (74%) | 0/51 |
| | FP-1/RP-1 | | | |

Of the five selected bands gel (a-e) of FIG. 3 extracted and sequenced, one band (band e, FIG. 3 and Table 1, Experiment 1) gave sequence homology to the target DNA (Litmus 28 plasmid) with 71 out of 84 identities using BLAST (bl2seq) (Tatusova et al., 1999). Of the four other samples sequenced, one gave homology to the Tn5 T7/KAN-2 transposon (band a in FIG. 3), which was most likely a double transposition of the HyperMu transposon into the Tn5 T7/KAN-2 transposon, while the remaining three bands did not give any sequence homology to the target. Since these particular transposons insert in random regions of the target, different lengths of amplicons will be generated from the different regions of target flanked by the two different transposons. The gel image in FIG. 3 is showing the bands that were produced as a result of PCR amplification of the excised (products from the transposition reaction) bands in FIG. 2. In FIG. 3, labels a, b, c, d and e are indicating which bands (PCR products) were excised/purified and sequenced.

Table 1, Experiment 2, reports the results of another double transposition experiment using the same transposons and targets as Table 1, Experiment 1, where 3 of 12 excised amplicons have Litmus 28 homology. In this second experiment, the 3 amplicons (Table 1, Experiments 2a-c (in reference to bands a-c in FIG. 3) were sequenced with both the HyperMu™ and Tn5 T7/KAN-2™ primer pairs in separate sequencing reactions and both primer sets gave Litmus 28 sequence. In particular, the results support double transposition having taken place as the two different transposon primer sets gave sequence homology to the target. The only means for this to happen on the same sample is if the target sequence is flanked by the two different transposons. Table 1 also lists the primer set used to sequence the band and the percent identity of the known sequence to what was achieved experimentally from the sequence reads. For each sequencing reaction only one primer pair was included. This is due to the fact that sequencing is a linear PCR reaction and does not require two primers. The two primers used per sequencing reaction run in opposite directions. This means that primers will bind to both strands of a transposon. One primer will run along the target while the other primer will not be able to extend past the priming site due to no available template.

The above results in connection with results illustrated in Example 2 demonstrate that Applicants have recovered, purified, and confirmed sequence for the amplified DNA, which originated from double transposition into target DNA through in vitro steps, without the need for transformation into bacteria.

Applicants have demonstrated a truly in vitro double transposition reaction, where the target DNA was then repaired in vitro, and used in a PCR reaction to generate amplicons, which were sequenced. As this approach inserts sequences to which universal sequencing primers can be applied, it is expected to be used for genetic discovery of unknown organisms and important metagenomic applications.

### Example 7

### Generation and Characterization of Transposition Through an Entirely in vitro Process v. Conventional Transposition

In addition to simplifying experimental procedures, time can be saved by conducting the reactions by the in vitro methods presented herein. Table 2 shows a time comparison in hours between the double transposition assay and the conventional use of transposons for sequencing unknown DNA. The various steps are listed for each system, as well as the approximate time required for performing each step. In vitro double transposition is considerably faster than the conventional use of transposition kits.

TABLE 2

| | Double | Conventional |
|---|---|---|
| | Transposition | Transposition |
| Transposition Reaction | 2 | 2 |
| Gel Electrophoresis/Extraction | 2 | N.A. |
| PreCR^{TM}/E.coli Polymerase I | 0.5 | N.A. |
| PCR | 2 | N.A. |
| ExoSAP-IT^{TM} treatment | 0.5 | N.A. |
| Transformation/plating | N.A. | 24 |
| Colony picking/culturing | N.A. | 24 |
| Miniprep overnight cultures | N.A. | 0.5 |
| Sequencing reactions/Capillary | 5 | 5 |
| electrophoresis | | |
| Estimated total time | ~12 hours | ~56 hours |

The results from Table 2 show a significant time savings gained by using the in vitro double transposition method for sequencing versus using concentration transposition prior to sequencing. Further time savings can be gained by using fast gel-electrophoresis systems that would be able to sufficiently separate the samples in less than 20 minutes. Using the fast electrophoresis in conjunction with the extraction protocol would drop the gel electrophoresis/extraction time from 2 hours down to 1 hr.

### Example 8

### Transposition in a High Throughput, Microfluidic System

Applicants have developed a method that can be performed entirely in vitro so that it can be incorporated into a high throughput, automated microfluidic system. While the full realization of the double transposition reaction in microdroplets will require additional fluidic capabilities so that reagents can be added to droplets sequentially, here Applicants demonstrate that single transposition can occur in high throughput systems. To date, transposition has not been demonstrated in emulsion format, microfluidic or otherwise. As part of the single transposition demonstration, it was important to show that transposition took place in the microfluidic droplets, rather than in the bulk sample prior to droplet formation. To show this, a time course study was undertaken in bulk format with the GPS 1.1 transposon and circular Litmus 28 target DNA.

A master mix of five times the volume of a single transposition reaction was prepared for the bulk and droplet reactions following the manufacturer's protocol for the GPS-1 Genome Priming System™, with the exception of adding BSA to the reaction. BSA was added to stabilize the enzymes in the aqueous droplets by minimizing the negative effects of the oil/surfactant interface on the enzyme. (Williams, et al., 2006; Beer, et al., 2007; Beer, et al., 2008).

For the GPS-1 system, the reactions contained 5 μl BSA at 10 mg/ml, 10 μl of 10×GPS buffer, 50 transprimer donor GPS 1.1 bearing a kanamycin resistance marker, 5 μl of Litmus 28 plasmid DNA (80 ng/μl), 650 water, and 5 μl of TnsABC* transposase. This mixture was incubated at 37° C. for 10 minutes and then 50 of start solution was added. For the HyperMu™ system, a 100 μl reaction contained 25 μl BSA at 10 mg/ml, 10 μl of 10× HyperMu™ reaction buffer, 10 μl HyperMu™ CHL-1> transposon, 50 of Litmus 28 plasmid DNA (80 ng/μl), 450 nuclease-free water, and 5 μl of Mu transposase. Circular Litmus 28 was used in these experiments so the plasmid could replicate in bacteria. The 2 hour EZTn5<T7/Kan-2> reactions were the same as the HyperMu™ formulation, with the exception of using 10 μl of EZ-Tn5 transposase and 5 μl of EZ-Tn5 transposon. The 100 μl samples were split with 20 μl going to the bench top reaction and 80 μl to the microfluidic droplet system. All reactions were carried out at room temperature (23.6° C.). Droplets were generated on chip using the system described previously (Tatusova and (1999)) with an aqueous stream flow rate of 0.9 μl/min and an oil stream flow rate of 5.0 μl/min.

A monodisperse stream of 50 μm (˜65 pl) aqueous droplets isolated in the oil carrier was produced and recovered from the chip through a capillary and captured in a microcentrifuge tube. A total of 300 μl of emulsion containing approximately 700,000 droplets was recovered from the chip in 85 minutes and was divided into 25 μl aliquots. Aliquots (3 μl) of the bulk reaction were made into separate tubes, and both the bulk and droplet aliquots were heat inactivated in a thermocycler at 75° C. for 10 minutes at time points of 0, 30, 60, 90, and 150 minutes post droplet collection. After heat inactivation, the droplet emulsion was broken by centrifugation at 13,000 RPM for 5 minutes, and aqueous portions were recovered by removing the upper oil layer and extracting with two rounds of 25 μl of water saturated diethyl ether. Of the 25 μl emulsion volume, typically 3 μl of aqueous phase were recovered after extraction.

To measure the extent of transposition, 3 μl of recovered aqueous reaction from the droplet emulsion and 3 μl of the bulk reaction were transformed into UltraMaxDH5α-FT™ (Invitrogen) competent cells using the heat shock method according to manufacturer's protocol, incubated at 37° C. for 1 hour, and plated on LB agar plates with appropriate antibiotics overnight. Two to three plate replicates were made for each time point in bulk and droplet format as described in the figure legends. Colonies were counted the next day for each time point of bulk and droplet reactions.

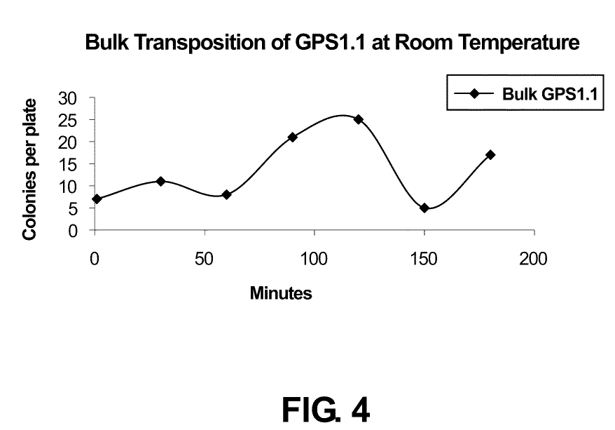

As shown in FIG. 4, the time course study showed characteristic fluctuations in the number of antibiotic resistant colonies that arose after transforming aliquots of the reaction over 30 minute intervals and demonstrated that the transposition reaction was still occurring two hours into the study, which would allow ample time to recover the emulsion from the microfluidic platform.

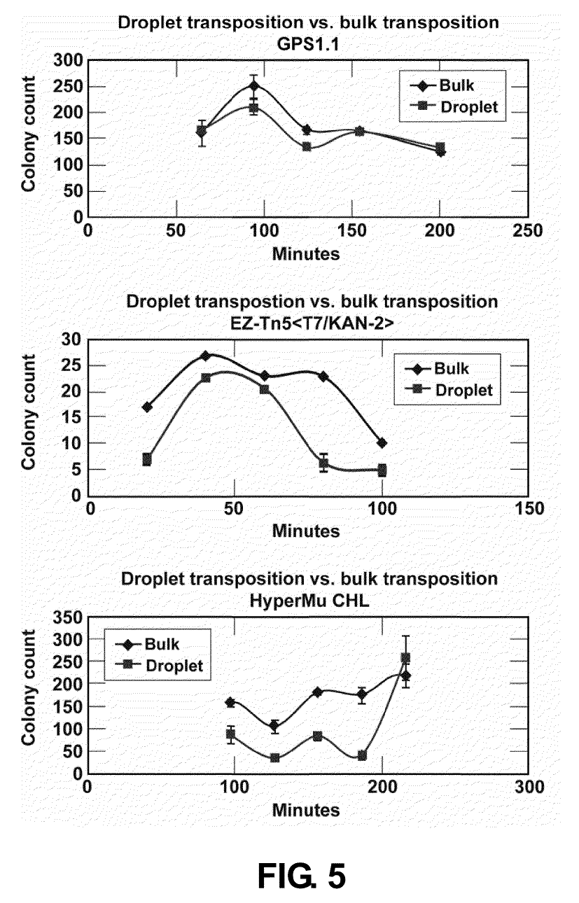

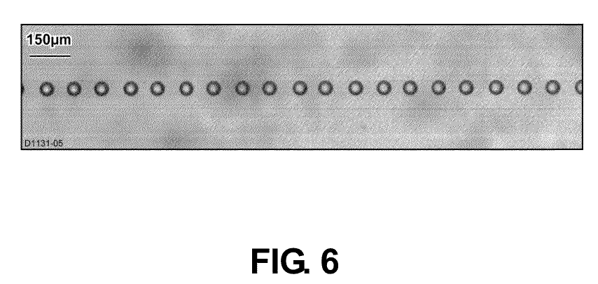

FIG. 5 illustrates a time course study that plots single transposition reactions occurring at room temperature simultaneously in both bulk and droplet formats for all three transposons described herein in the materials and methods. The temporal response shows characteristic increases and decreases in the number of colonies that form, with patterns that roughly track one another. In each system, the pattern of observed colony count continues to fluctuate with time, suggesting that transposition continues after the reactants have become emulsified, demonstrating that transposition can be carried out in droplet based systems. The differences in initial time points in the three transposon systems varied due to experimental factors, such as reagent loading and fluid processing speed. The emulsions consisted of highly monodisperse droplets, as shown in FIG. 6.

The droplet transposition results above indicate that single transposition occurs in vitro in droplet format, and exhibits similar colony count dynamics to the bulk transposition. Additionally, these results provide evidence of transposition occurring in picoliter-sized droplets and provide a means for a high throughput, automated microfluidic system.

When paired with emerging droplet-based technologies, such as sample preparation techniques that can sort cells from viruses (Jung, K et al. (2008)) and techniques of droplet merging, which will allow the addition of sequencing primers and other reagents to the transposon-derived amplicons, the presented methods and systems will allow a complete in vitro target identification. This will be accomplished by isolating and amplifying genetic material in droplets, prior to sequencing the droplets' contents. The double transposition method established herein provides a novel approach to sequencing and identifying unknown nucleic acids.

Transfer of the transposition assay herein exemplified from benchtop to the microfluidic system involves creating a mastermix that is at least 5×'s the volume of the benchtop double transposition reaction and with the addition of BSA to help alleviate damage to the enzyme from oil/surface interactions. This mastermix can then be loaded onto the microfluidic system followed by the mastermix/oil dispersion process in order to create picoliter sized reaction cells. Since the double transposition reaction occurred at 37° C. on the benchtop, one would also be able to heat the microfluidic system to this temperature as well. However, results from the single transposition reactions demonstrate that the reactions can be carried out at room temperature. Droplets can be collected and heat inactivated on the benchtop, if not heat inactivated on the instrumentation.

The examples set forth above are provided to give those of ordinary skill in the art a complete disclosure and description of how to make and use the embodiments of the transposons, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

The entire disclosure of each document cited (including patents, patent applications, journal articles, abstracts, laboratory manuals, books, or other disclosures) in the Background, Summary, Detailed Description, and Examples is hereby incorporated herein by reference.

It is to be understood that the disclosures are not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

Although any methods and materials similar or equivalent to those described herein can be used in the practice for testing of the specific examples of appropriate materials and methods are described herein.

A number of embodiments of the disclosure have been described. Nevertheless, it will be understood that various modifications can be made without departing from the spirit and scope of the present disclosure. Accordingly, other embodiments are within the scope of the following claims.

### REFERENCES

1 B. McClintock, The Origin and Behavior of Mutable Loci in Maize. Proc Natl Acad Sci U S A 36 (1950) 344-55.
2. B. McClintock, Induction of instability at selected loci in maize. Genetics 38 (1953) 579-99.
3. R. A. Jilk, D. York, and W. S. Reznikoff, The organization of the outside end of transposon Tn5. J Bacteriol. 178 (1996) 1671-9.
4. N. L. Craig, Target Site Selection in Transposition. Annu. Rev. Biochem. 66 (1997) 437-74.
5. R. T. DeBoy, and N. L. Craig, Tn7 transposition as a probe of cis interactions between widely separated (190 kilobases apart) DNA sites in the Escherichia coli chromosome. J. Bacteriol. 178 (1996) 6184-91.
6. A. E. Stellwagen, and N. L. Craig, Avoiding self: two Tn7-encoded proteins mediate target immunity in Tn7 transposition. EMBO J. 16 (1997) 6823-34.
(7) M. Margulies, M. Egholm, W. E. Altman, S. Attiya, J. S. Bader, L. A. Bemben, J. Berka, M. S. Braverman, Y. J. Chen, Z. Chen, S. B. Dewell, L. Du, J. M. Fierro, X. V. Gomes, B. C. Godwin, W. S. Helgesen, C. H. Ho, G. P. Irzyk, S. C. Jando, M. L. Alenquer, T. P. Jarvie, K. P. Jirage, J. B. Kim, J. R. Knight, J. R., Lanza, J. H. Leamon, S. M. Lefkowitz, M. Lei, J. Li, K. L. Lohman, H. Lu, V. B. Makhijani, K. E. McDade, M. P. McKenna, E. W. Myers, E. Nickerson, J. R. Nobile, R. Plant, B. P. Puc, M. T. Ronan, G. T., Roth, G. J. Sarkis, J. F. Simons, J. W. Simpson, M. Srinivasan, K. R. Tartaro, A. Tomasz, K. A. Vogt, G. A., Volkmer, S. H. Wang, Y. Wang, M. P. Weiner, P. Yu, R. F. Begley, and J. M. Rothberg, Genome sequencing in microfabricated high-density picolitre reactors. Nature 437 (2005) 376-80.
(8) A. W. Martinez, S. T. Phillips, B. J. Wiley, M. Gupta and G. M. Whitesides. FLASH: A rapid method for prototyping paper-based microfluidic devices. Lab Chip, (2008) 8, 2146-2150
9. Marcus, J. M. Female Site-Specific Transposase-Induced Recombination: A High-Efficiency Method for Fine Mapping Mutations on the X Chromosome in Drosophila. Genetics, 163, 591-597, (2003).
10. T. A. Tatusova, and T. L. Madden, BLAST 2 SEQUENCES, a new tool for comparing protein and nucleotide sequences. FEMS Microbiol. Lett. 174 (1999) 247-50.
11. R. Williams, S. G. Peisajovich, O. J. Miller, S. Magdassi, D. S. Tawfik, D. S. and A. D. Griffiths, A. D. Amplification of complex gene libraries by emulsion PCR. Nat. Methods 3 (2006) 545-50.

12. N. R. Beer, B. J. Hindson, E. K. Wheeler, S. B. Hall, K. A. Rose, I. M. Kennedy, and B. W. Colston, On-chip real time single copy polymerase chain reaction in picoliter droplets. Anal. Chem. 79 (2007) 8471-5.
13. N. R. Beer, E. K. Wheeler, L. Lee-Houghton, N. Watkins, S. Nasarabadi, N. Hebert, P. Leung, D. W. Arnold, C. G. Bailey, and B. W. Colston, On-Chip Single-Copy Real-Time Reverse-Transcription PCR in Isolated Picoliter Droplets. Anal. Chem. 80 (2008) 1854-8.
14. B. Jung, K. Fisher, K. D. Ness, K. A. Rose, and R. P. Mariella, Jr. Acoustic particle filter with adjustable effective pore size for automated sample preparation. Anal. Chem. (2008) 80, 8447-52.

What is claimed is:

1. A method to introduce two or more functional polynucleotides into a target polynucleotide, the method comprising
inserting at least one first functional polynucleotide of the two or more functional polynucleotides into the target polynucleotide within a first transponson; and
inserting at least one second functional polynucleotide of the two or more functional polynucleotides into the target polynucleotide within a second transposon,
wherein the first functional polynucleotide and the second functional polynucleotide are operably connected.

2. The method of claim 1, further comprising repairing the target polynucleotide following at least one of inserting the at least one first polynucleotide and inserting the at least one second functional polynucleotide.

3. The method of claim 2, wherein the repairing is performed in vitro.

4. The method of claim 1, wherein at lest one of inserting at least one first polynucleotide and inserting at least one second polynucleotide is preceded by digesting the target polynucleotide at specific or random sites to increase efficiency of the inserting and/or to control location of insertion for at least one of the transposons.

5. The method of claim 1, wherein inserting at least one first functional polynucleotide is performed by contacting the at least one first transposon with the target sequence in presence of first one or more transposases; and wherein inserting at least one second functional polynucleotide is performed by contacting the second transposon with the target sequence in presence of second one ore more transposases.

6. The method of claim 5, wherein the first one or more transposases and the second one or more transposases catalyze site specific transposition.

7. The method of claim 5, wherein the first one or more transposases and the second one or more transposases catalyze random transposition.

8. The method of claim 5, wherein the first one or more transposases and the second one or more transposases are tethered together.

9. A method to introduce two or more functional polynucleotides into a target polynucleotide, the method comprising
inserting the two or more functional polynucleotides into the target polynucleotide with two or more transposons, each of the two or more functional polynucleotides comprised within each of the two or more transposons, the inserting resulting in a targeted polynucleotide containing the two or more functional polynucleotides; and
repairing the targeted product in vitro.

10. The method of claim 9, wherein the repairing is performed by contacting the targeted product with one or more enzymes, and suitable reagents to digest overhangs of the transposon and polymerizing a complementary strand for the single stranded target polynucleotide.

11. The method of claim 10, wherein the enzyme and suitable reagents comprise a polymerase with exonuclease activity.

12. A system for introducing two or more functional polynucleotides into a target polynucleotide, the system comprising
at least a first transposon comprising at least a first functional polynucleotide; and
at least a second transposon comprising at least a second functional polynucleotide, wherein the at least one first functional polynucleotide and the at least one second polynucleotide are operably connected.

13. The system of claim 12, further comprising reagents suitable to repair overhangs and gaps in transposon insertion sites.

14. The system of claim 13, wherein the reagents suitable to repair overhangs and gaps comprise a polymerase with exonuclease activity.

15. A method for in vitro amplification of a target polynucleotide comprised between a first and a second region flanking the target polynucleotide, the method comprising
inserting a first functional polynucleotide into the first region within a first transponson;
inserting a second functional polynucleotide into the second region within a second transponson;
repairing in vitro a targeted polynucleotide comprising at least one of the inserted first functional polynucleotide and the inserted second functional polynucleotide; and
amplifying the target polynucleotide using at least one pair of primers, each pair comprising a first primer able to hybridize the first functional polynucleotide and a second primer able to hybridize the second functional polynucleotide.

16. The method of claim 15, wherein at lest one of inserting a first polynucleotide and inserting a second polynucleotide is preceded by digesting the target polynucleotide at specific or random sites to increase efficiency of the inserting and/or to control location of insertion for at least one of the transposons.

17. The method of claim 15, wherein the amplifying is performed by using two pairs of primers.

18. The method of claim 15, wherein one or more of inserting a first functional polynucleotide, inserting a second functional polynucleotide, repairing the target polynucleotide, and amplifying the target polynucleotide is performed within a microfluidic or robotic arrangement utilizing microwells, microarrays, or micro-titer plates configured to allow sample discretization and transposon operation on volumes of any desirable scale.

19. The method of claim 15, further comprising optically detecting the target polynucleotide following at least one of inserting a first functional polynucleotide, and inserting a second functional polynucleotide in droplets or compartments utilizing functional probes and subsequently selecting targeted polynucleotide for additional repair, hybridization, and/or amplification.

20. A system for in vitro amplification of a target polynucleotide comprised between a first and a second region flanking the target polynucleotide, the system comprising
a first transposon comprising a first functional polynucleotide,
a second transposon comprising a second functional polynucleotide; and
at least one pair of primers, each pair comprising a first primer able to hybridize the first functional polynucleotide and a second primer able to hybridize the second functional polynucleotide.

21. The system of claim 20, further comprising reagents suitable to perform repairing of a targeted polynucleotide.

22. The system of claim 21, wherein the reagents are comprised on magnetic or non-magnetic beads,

23. The system of claim 20, wherein at least one of the first transposon, second transposons and pair of primers is comprised in a monodisperse and/or a polydisperse emulsion.
